(19) United States
(12) Patent Application Publication
(10) Pub. No.: US 2025/0076287 A1
(43) Pub. Date: Mar. 6, 2025

(54) PERFORMING MEASUREMENTS ON A SAMPLE

(71) Applicant: S.D. Sight Diagnostics Ltd., Tel Aviv (IL)

(72) Inventors: Amir ZAIT, Binyamina (IL); Arnon HOURI YAFIN, Jerusalem (IL); Dan GLuck, Kadima (IL); Sharon PECKER, Rehovot (IL); Yochay Shlomo ESHEL, Sde Warburg (IL); Sarah LEVYS CHREIER, Jaffa (IL); Joseph Joel POLLAK, Neve Daniel (IL)

(73) Assignee: S.D. Sight Diagnostics Ltd., Tel Aviv (IL)

(21) Appl. No.: 18/951,911

(22) Filed: Nov. 19, 2024

Related U.S. Application Data
(63) Continuation of application No. 17/082,615, filed on Oct. 28, 2020, now Pat. No. 12,174,175.
Application No. 17/082,615 is a continuation of application No. 16/099,270, filed on Nov. 6, 2018, now Pat. No. 11,099,175.
(60) Provisional application No. 62/334,517, filed on May 11, 2016.

(51) Int. Cl. G01N 33/50; G01N 15/01; G01N 15/06; G01N 33/49; G01N 33/569; G01N 33/72

(52) U.S. Cl. CPC G01N 33/5094; G01N 15/06; G01N 33/49; G01N 33/56972; G01N 33/721; G01N 2015/012; G01N 2015/016

(57) ABSTRACT

Apparatus and methods are described including measuring a measurand within a first portion of a bodily sample, measuring relative amounts of first and second components within a second portion of the bodily sample that is diluted relative to the first portion of the bodily sample, and determining a parameter of the bodily sample based upon the measurand within the first portion of the bodily sample and the relative amounts of first and second components within the second portion of the bodily sample. Other applications are also described.

### CROSS-REFERENCE TO RELATED APPLICATIONS

The present application is a continuation of U.S. Ser. No. 17/082,615 to Zait, filed Oct. 28, 2020 (published as US 2021/0041422), which is a continuation of U.S. Ser. No. 16/099,270 to Zait, filed Nov. 6, 2018 (issued as U.S. Pat. No. 11,099,175), which is a US national phase application of PCT Application No. PCT/IL2017/050526 to Zait (published as WO 17/195208), filed May 11, 2017, which claims priority from U.S. Provisional Patent Application No. 62/334,517 to Zait, filed May 11, 2016, entitled “Method and Apparatus for Estimating Dilution and Concentration.”

The above-referenced applications are incorporated herein by reference.

### FIELD OF EMBODIMENTS OF THE INVENTION

Some applications of the presently disclosed subject matter relate generally to analyzing a biological sample, and in particular, to analyzing a blood sample by performing optical measurements.

### BACKGROUND

Several methods exist for quantifying parameters in a sample (such as a blood sample). In some such methods, the sample is diluted before being analyzed. For example, a blood sample may be diluted in order to increase visibility of components of the sample within microscopic images of the sample, and/or staining substances may be added to the blood sample, in order to stain given components within the sample.

In some cases, samples are analyzed using more than one type of measuring device. For example, a microscope is sometimes used in order to analyze individual cells within the sample, whereas imaging devices, such as spectral cameras, are used to analyze the sample on a bulk level (e.g., by performing optical absorption, transmittance, fluorescence, and/or luminescence measurements).

### SUMMARY OF EMBODIMENTS

In accordance with some applications of the present invention, a portion of a blood sample is diluted using a dilution technique, such as a technique as described in US 2015/0316477 to Pollak, which is incorporated herein by reference. The blood sample portion is typically imaged using a microscope system (which may be manual or automated). For some applications, the microscope images are analyzed (e.g. manually, or using a computer processor that runs suitable computer software) to identify different blood cells.

For some applications of the present invention, variation and/or errors that occur in a dilution process are accounted for. Typically, an error of 10 percent in the dilution factor may correspond directly to a 10 percent error in the count of, for example, red blood cells per unit volume (e.g., per microliter) of blood. Such errors in dilution can originate from a number of sources. Illustrative examples of sources of such errors (which are not intended to limit the scope of the present invention) include pipetting inaccuracy or error, calibration inaccuracy or error, mixing inaccuracy or error, etc. Therefore, in accordance with some applications of the present invention, a measurement is made on a source sample portion (e.g., an undiluted blood sample portion) from which the diluted sample portion is extracted. This measurement typically corresponds to at least one of the measurements measured on the diluted sample portion. For example, the measurement performed on the source sample portion may include measurement of: hemoglobin content, white blood cell content, red blood cell content, hematocrit, content of a specific white blood cell type, platelet content, and/or any measurand that is measured or that can be inferred for the diluted sample portion. For some applications, a normalization factor is determined, the normalization factor being a property of the source sample portion to which other measurements are correlated (e.g. the number of red blood cells per unit area or per unit volume in the source sample portion). Typically, measurands within the sample (e.g., within the source sample portion) are measured based upon the normalization factor, as described in further detail hereinbelow.

For some applications, hematocrit is measured by performing a first measurement on a blood sample, and mean corpuscular volume within the blood sample is measured, by performing a second measurement on the blood sample. For example, hematocrit may be measured using the micro-hematocrit method (in which the blood is centrifuged), and or by performing ultrasonic and/or impedance measurements on a first portion of the blood sample, and the mean corpuscular volume may be measured by analyzing microscopic images that are acquired of a second portion of the blood sample. Typically, the second portion of the sample is diluted with respect to the first portion of the blood sample, e.g. in order to improve visibility of the individual cells, for the purpose of staining the second portion of the sample, and/or for a different reason. For some applications, based on the relationship between the hematocrit and the mean corpuscular volume, a relationship between the first portion of the sample and second portion of the sample is determined. For some applications, a parameter of the source sample portion is determined, based on the relationship between the hematocrit and the mean corpuscular volume. Typically, the red blood cell count (e.g., count per unit volume) within the sample is determined by dividing the hematocrit by the mean corpuscular volume. For some applications, counts of one or more additional components within the sample (e.g., red blood cells of a given type, white blood cells, white blood cells of a given type, circulating tumor cells, platelets, platelets of a given type, bacteria, pathogens, pathogens of a given type, reticulocytes, and/or Howell-Jolly bodies) are determined, based on the red blood cell count within the sample. For example, a ratio between the red blood cell count and the counts of the one or more additional components within a portion of the sample may be determined by analyzing a microscopic image of the portion of the sample. The counts of the one or more additional components may then be determined, based on the red blood cell count within the sample and the ratio between the red blood cell count and the counts of the one or more additional components within the portion of the sample.

For some applications, hemoglobin concentration is measured by performing a first measurement on a blood sample, and mean corpuscular hemoglobin within the blood sample is measured, by performing a second measurement on the blood sample. For example, hemoglobin concentration may be measured by performing optical density measurements on a first portion of the blood sample, and the mean corpuscular hemoglobin may be measured by analyzing microscopic images that are acquired of a second portion of the blood sample. Typically, the second portion of the sample is diluted with respect to the first portion of the blood sample, e.g. in order to improve visibility of the individual cells, and/or for the purpose of staining the second portion of the sample, and/or for a different reason. For some applications, based on the relationship between the hemoglobin concentration and the mean corpuscular hemoglobin, a relationship between the first portion of the sample and second portion of the sample is determined. For some applications, a parameter of the source sample portion is determined, based on the relationship between the hemoglobin concentration and the mean corpuscular hemoglobin. Typically, the red blood cell count (e.g., count per unit volume) within the sample is determined by dividing the hemoglobin concentration by the mean corpuscular hemoglobin. For some applications, counts of one or more additional components within the sample (e.g., red blood cells of a given type, white blood cells, white blood cells of a given type, circulating tumor cells, platelets, platelets of a given type, bacteria, pathogens, pathogens of a given type, reticulocytes, and/or Howell-Jolly bodies) are determined, based on the red blood cell count within the sample. For example, a ratio between the red blood cell count and the counts of the one or more additional components within a portion of the sample may be determined, by analyzing a microscopic image of the portion of the sample. The counts of the one or more additional components may then be determined, based on the red blood cell count within the sample and the ratio between the red blood cell count and the counts of the one or more additional components within the portion of the sample.

For some applications of the present invention, two or more measurements (which are typically optical measurements) are performed upon a biological sample. Typically, the biological sample is a blood sample. For some applications, a bulk-level measurand of the sample is measured, by performing a first measurement on the sample, and a cellular-level measurand of the sample is measured, by performing a second measurement on the sample. For the purpose of the present applications, the term “cellular-level measurand” should be understood to mean a measurand that relates to one or more parameters of individual cells or other non-dissolved components within the sample, such as, mean corpuscular volume, mean corpuscular hemoglobin, mean platelet volume, and/or red blood cell distribution width, etc. Measurement of a cellular-level measurand typically involves a first step of identifying individual cells or other non-dissolved components within the sample (e.g., identifying such components within a microscopic image), and a second step of identifying a parameter of such individual identified components. Typically, a cellular-level measurand is measured by analyzing one or more microscopic images of the sample. For the purpose of the present applications, the term “bulk-level measurand” should be understood to mean a measurand that relates a parameter of the sample as a whole, and that does not require the two steps of identifying individual cells or other non-dissolved components within the sample, and identifying a parameter of such individual identified components. For example, such a measurand may include the optical density of a given component (which is measured by performing a measurement on a bulk volume of the sample, e.g., even after performing lysis of individual components within the bulk volume), a count per unit volume of a given component (which is typically measured by identifying such components, but does not require identifying a parameter of individual identified components), and/or the concentration of a given component (such as, red blood cell concentration, hemoglobin concentration, white blood cell concentration, platelet concentration, and/or hematocrit). Typically, bulk-level measurands are measured by performing a measurement on a bulk volume of the sample. For example, such measurements may include ultrasonic, impedance, optical absorption, transmittance, fluorescence, microscopic and/or luminescence measurements that are performed on a bulk volume of the sample. Typically, a parameter of the sample is determined, based on a relationship between the bulk-level measurand and the cellular-level measurand.

For some applications, first and second optical measurements are performed on a sample, using one or more optical measurement devices under respective sets of measuring conditions that are different from each other. A measurand of the sample is measured based upon the first optical measurement, and a measurand of the sample is measured based upon the second optical measurement. In accordance with respective applications, the measurand that is measured based upon the second optical measurement is the same as the measurand that is measured based upon the first optical measurement, or is different from the measurand that is measured based upon the first optical measurement. In accordance with respective applications, the first and second optical measurements are performed on the same portion of the sample, or on different portions of the sample. For some applications, one of the optical measurements is performed on a portion of the sample that is diluted with respect to a portion of the sample upon which the other optical measurement is performed.

Typically, based on a relationship between the measurand measured based upon the first optical measurement and the measurand measured based upon the second optical measurement, a relationship between the measuring conditions of the one or more optical measurement devices that were used to perform the first and second optical measurements is determined. For example, the first and second optical measurements may be performed on respective portions of the sample that are disposed in respective portions of one or more sample chambers having respective dimensions (e.g., respective heights). For some such applications, a relationship between dimensions of the respective portions of the one or more sample chambers is determined, based on the relationship between the measurand measured based upon the first optical measurement and the measurand measured based upon the second optical measurement. Alternatively or additionally, based on the relationship between the measurand measured based upon the first optical measurement and the measurand measured based upon the second optical measurement, the field of view from which one of the first and second optical measurements (e.g., a microscopic image) was measured is determined, and/or the level of magnification at which one of the first and second optical measurements (e.g., a microscopic image) was measured is determined. For some applications, the first and second measurements are normalized with respect to one another. Subsequently, a parameter of the sample is determined based upon the normalization of the first and second measurements with respect to one another.

There is therefore provided, in accordance with some applications of the present invention, a method for use with a blood sample, the method including:
measuring hemoglobin concentration within at least a portion of the blood sample, by performing a first measurement on the blood sample;
measuring mean corpuscular hemoglobin in the blood sample, by performing a second measurement on the blood sample; and
determining a parameter of the blood sample, based on a relationship between the concentration of hemoglobin and the mean corpuscular hemoglobin.

In some applications, determining the parameter of the blood sample includes normalizing the first and second measurements with respect to each other, based on the relationship between the hemoglobin concentration and the mean corpuscular hemoglobin.

In some applications, performing the first measurement on the blood sample includes performing an optical density measurement on the blood sample.

In some applications, measuring hemoglobin concentration within at least the portion of the blood sample includes measuring the hemoglobin concentration within a first portion of the blood sample, measuring mean corpuscular hemoglobin in the blood sample includes measuring mean corpuscular hemoglobin within a second portion of the blood sample, and determining the parameter of the sample includes determining a relationship between the first portion of the sample and second portion of the sample, based on the relationship between the hemoglobin concentration and the mean corpuscular hemoglobin.

In some applications, determining the parameter of the sample includes determining a count of a component of the blood selected from the group consisting of: red blood cells, red blood cells of a given type, white blood cells, white blood cells of a given type, circulating tumor cells, platelets, platelets of a given type, bacteria, pathogens, pathogens of a given type, reticulocytes, and Howell-Jolly bodies.

In some applications, determining the parameter of the sample includes determining a concentration of a component of the blood selected from the group consisting of: hemoglobin, red blood cells, red blood cells of a given type, white blood cells, white blood cells of a given type, circulating tumor cells, platelets, platelets of a given type, bacteria, pathogens, pathogens of a given type, reticulocytes, and Howell-Jolly bodies.

In some applications, determining the parameter of the sample includes determining a hematocrit of the sample.

In some applications, measuring the hemoglobin concentration includes measuring the hemoglobin concentration within a first portion of the blood sample, and measuring mean corpuscular hemoglobin in the blood sample includes measuring mean corpuscular hemoglobin within a second portion of the blood sample that is diluted with respect to the first portion of the blood sample.

In some applications, determining the parameter of the blood sample includes determining a normalization factor by determining a property of the first portion of the sample portion for using as a reference to which measurements within the second portion can be correlated.

In some applications, determining the parameter of the blood sample, includes determining a red blood cell count within the sample, by dividing the hemoglobin concentration by the mean corpuscular hemoglobin.

In some applications, determining the parameter of the blood sample, further includes determining counts of one or more components within the sample, based on the red blood cell count within the sample.

In some applications, determining the counts of one or more components within the sample, includes:
determining a ratio between the red blood cell count and the counts of the one or more components within a portion of the sample, by analyzing a microscopic image of the portion of the sample, and
determining the count of the one or more components based on the red blood cell count within the sample and the ratio between the red blood cell count and the counts of the one or more components within the portion of the sample.

There is further provided, in accordance with some applications of the present invention, apparatus for use with a blood sample, the apparatus including:
at least one computer processor configured to:
   measure hemoglobin concentration within at least a portion of the blood sample, by performing a first measurement on the blood sample,
   measure mean corpuscular hemoglobin in the blood sample, by performing a second measurement on the blood sample, and
   determine a parameter of the blood sample, based on a relationship between the concentration of hemoglobin and the mean corpuscular hemoglobin.

There is further provided, in accordance with some applications of the present invention, a computer software product, for use with a blood sample, the computer software product including a non-transitory computer-readable medium in which program instructions are stored, which instructions, when read by a computer cause the computer to perform the steps of:
measuring hemoglobin concentration within at least a portion of the blood sample, by performing a first measurement on the blood sample;
measuring mean corpuscular hemoglobin in the blood sample, by performing a second measurement on the blood sample; and
determining a parameter of the blood sample, based on a relationship between the concentration of hemoglobin and the mean corpuscular hemoglobin.

There is further provided, in accordance with some applications of the present invention, a method for use with a blood sample, the method including:
measuring hematocrit in the blood sample, by performing a first measurement on the blood sample;
measuring mean corpuscular volume in the blood sample, by performing a second measurement on the blood sample; and
determining a parameter of the blood sample, based on a relationship between the hematocrit and the mean corpuscular volume.

In some applications, determining the parameter of the blood sample includes normalizing the first and second measurements with respect to each other, based on the relationship between the hematocrit and the mean corpuscular volume.

In some applications, performing the first measurement on the blood sample includes performing a measurement on the blood sample selected from the group consisting of: an ultrasonic measurement, and an impedance measurement.

In some applications, performing the first measurement on the blood sample includes centrifuging the blood sample.

In some applications, performing the second measurement includes performing the second measurement by analyzing a microscopic image of a portion of the blood sample.

In some applications, measuring the hematocrit includes measuring the hematocrit on a first portion of the blood sample, measuring mean corpuscular volume in the blood sample includes measuring mean corpuscular volume upon a second portion of the blood sample, and determining the parameter of the sample includes determining a relationship between the first portion of the sample and second portion of the sample, based on the relationship between the hematocrit and the mean corpuscular volume.

In some applications, determining the parameter of the sample includes determining a count of a component of the blood selected from the group consisting of: red blood cells, red blood cells of a given type, white blood cells, white blood cells of a given type, circulating tumor cells, platelets, platelets of a given type, bacteria, pathogens, pathogens of a given type, reticulocytes, and Howell-Jolly bodies.

In some applications, determining the parameter of the sample includes determining a concentration of a component of the blood selected from the group consisting of: hemoglobin, red blood cells, red blood cells of a given type, white blood cells, white blood cells of a given type, circulating tumor cells, platelets, platelets of a given type, bacteria, pathogens, pathogens of a given type, reticulocytes, and Howell-Jolly bodies.

In some applications, measuring the hematocrit includes measuring the hematocrit on a first portion of the blood sample, and measuring mean corpuscular volume in the blood sample includes measuring mean corpuscular volume upon a second portion of the blood sample that is diluted with respect to the first portion of the blood sample.

In some applications, determining the parameter of the blood sample includes determining a normalization factor by determining a property of the first portion of the sample portion for using as a reference to which measurements within the second portion can be correlated.

In some applications, determining the parameter of the blood sample includes determining a red blood cell count within the sample by dividing the hematocrit by the mean corpuscular volume.

In some applications, determining the parameter of the blood sample further includes determining counts of one or more components within the sample, based on the red blood cell count within the sample.

In some applications, determining the counts of one or more components within the sample, includes:
determining a ratio between the red blood cell count and the counts of the one or more components within a portion of the sample, by analyzing a microscopic image of the portion of the sample, and
determining the count of the one or more components based on the red blood cell count within the sample and the ratio between the red blood cell count and the counts of the one or more components within the portion of the sample.

There is further provided, in accordance with some applications of the present invention, apparatus for use with a blood sample, the apparatus including:
at least one computer processor configured to:
   measure hematocrit in the blood sample, by performing a first measurement on the blood sample,
   measure mean corpuscular volume in the blood sample, by performing a second measurement on the blood sample, and
   determine a parameter of the blood sample, based on a relationship between the hematocrit and the mean corpuscular volume.

There is further provided, in accordance with some applications of the present invention, a computer software product, for use with a blood sample, the computer software product including a non-transitory computer-readable medium in which program instructions are stored, which instructions, when read by a computer cause the computer to perform the steps of:
measuring hematocrit in the blood sample, by performing a first measurement on the blood sample;
measuring mean corpuscular volume in the blood sample, by performing a second measurement on the blood sample; and
determining a parameter of the blood sample, based on a relationship between the hematocrit and the mean corpuscular volume.

There is further provided, in accordance with some applications of the present invention, a method for use with a first portion of a blood sample and a second portion of the blood sample that is diluted with respect to the first portion of the blood sample, the method including:
measuring relative amounts of first and second components within the first portion of the blood sample;
measuring a measurand within the second portion of the blood sample; and
determining a parameter of the blood sample based upon a relationship between the relative amounts of first and second components within the first portion of the blood sample, and the measurand within the second portion of the blood sample.

In some applications, measuring relative amounts of first and second components within the first portion of the blood sample includes analyzing a microscopic image of the first portion of the blood sample.

In some applications, measuring relative amounts of first and second components within the first portion of the blood sample includes measuring relative amounts of at least two components within the first portion of the blood sample, the two components being selected from the group consisting of: all white blood cell types, neutrophils, eosinophils, basophils, lymphocytes, monocytes, and white blood cell precursors.

In some applications, measuring relative amounts of first and second components within the first portion of the blood sample includes measuring relative amounts of at least two components within the first portion of the blood sample, the two components being selected from the group consisting of: red blood cells, reticulocytes, intracellular bodies, red blood cells having a given morphology, and Howell-Jolly bodies.

In some applications, measuring relative amounts of first and second components within the first portion of the blood sample includes measuring relative amounts of given types of platelets within the first portion of the blood sample.

In some applications, measuring the measurand within the second portion of the sample includes measuring an absolute count of cells of a given type within the second portion of the blood sample.

In some applications, measuring the measurand within the second portion of the sample includes measuring a concentration of a given component within the second portion of the blood sample.

In some applications, measuring the measurand within the second portion of the sample includes performing a bulk-level measurement upon the second portion of the blood sample.

There is further provided, in accordance with some applications of the present invention, apparatus for use with a blood sample, the apparatus including:
at least one computer processor configured to:
   measure relative amounts of first and second components within the first portion of the blood sample,
   measure a measurand within the second portion of the blood sample, and
   determine a parameter of the blood sample based upon a relationship between the relative amounts of first and second components within the first portion of the blood sample, and the measurand within the second portion of the blood sample.

There is further provided, in accordance with some applications of the present invention, a computer software product, for use with a blood sample, the computer software product including a non-transitory computer-readable medium in which program instructions are stored, which instructions, when read by a computer cause the computer to perform the steps of:
measuring relative amounts of first and second components within the first portion of the blood sample;
measuring a measurand within the second portion of the blood sample; and
determining a parameter of the blood sample based upon a relationship between the relative amounts of first and second components within the first portion of the blood sample, and the measurand within the second portion of the blood sample.

There is further provided, in accordance with some applications of the present invention, a method for use with a biological sample, the method including:
measuring a bulk-level measurand of the sample, by performing a first measurement on the sample;
measuring a cellular-level measurand of the sample, by performing a second measurement on the sample; and
determining a parameter of the sample, based on a relationship between the bulk-level measurand and the cellular-level measurand.

In some applications, determining the parameter of the blood sample includes normalizing the first and second measurements with respect to each other, based on the relationship between the bulk-level measurand and the cellular-level measurand.

In some applications, measuring the bulk-level measurand includes determining an optical density of a given component within the sample.

In some applications, measuring the cellular-level measurand includes analyzing a microscopic image of the sample.

In some applications, performing the first measurement on the sample includes performing the first measurement on the sample using a first set of measuring conditions, performing the second measurement on the sample includes performing the second measurement on the sample using a second set of measuring conditions, and determining the parameter of the sample includes determining a relationship between the measuring conditions that were used to perform the first and second measurements, based on the relationship between the bulk-level measurand and the cellular-level measurand.

In some applications, performing the first measurement includes performing the first measurement on a first portion of the sample, and performing the second measurement includes performing the second measurement upon the first portion of the sample.

In some applications, performing the first measurement includes performing the first measurement on a first portion of the sample, and performing the second measurement includes performing the second measurement upon a second portion of the sample that is different from the first portion of the sample. In some applications, determining the parameter of the sample includes determining a relationship between the first portion of the sample and second portion of the sample, based on the relationship between the bulk-level measurand and the cellular-level measurand. In some applications, performing the second measurement upon the second portion of the sample includes performing the second measurement upon a second portion of the sample that is diluted with respect to the first portion of the sample. In some applications, determining the parameter of the sample includes determining a normalization factor by determining a property of the first portion of the sample portion for using as a reference to which measurements within the second portion can be correlated. In some applications, determining the parameter of the sample includes determining a dilution ratio by which the second portion of the sample is diluted with respect to the first portion of the sample.

In some applications, the biological sample includes a blood sample, and determining the parameter of the sample includes determining a parameter of the blood sample.

In some applications:
measuring the bulk-level measurand of the sample includes measuring hematocrit of the blood sample;
measuring the cellular-level measurand of the sample includes measuring mean corpuscular volume of the blood sample; and
determining the parameter of the sample includes determining the parameter of the sample, based on a relationship between the hematocrit and the mean corpuscular volume.

In some applications:
measuring the bulk-level measurand of the sample includes measuring hemoglobin concentration within at least a portion of the blood sample;
measuring the cellular-level measurand of the sample includes measuring mean corpuscular hemoglobin of the blood sample; and
determining the parameter of the sample includes determining the parameter of the sample, based on a relationship between the hemoglobin concentration and the mean corpuscular hemoglobin.

There is further provided, in accordance with some applications of the present invention, apparatus for use with a biological sample, the apparatus including:
at least one computer processor configured to:
   measure a bulk-level measurand of the sample, by performing a first measurement on the sample,
   measure a cellular-level measurand of the sample, by performing a second measurement on the sample, and
   determine a parameter of the sample, based on a relationship between the bulk-level measurand and the cellular-level measurand.

There is further provided, in accordance with some applications of the present invention, a computer software product, for use with a biological sample, the computer software product including a non-transitory computer-readable medium in which program instructions are stored, which instructions, when read by a computer cause the computer to perform the steps of:
measuring a bulk-level measurand of the sample, by performing a first measurement on the sample;
measuring a cellular-level measurand of the sample, by performing a second measurement on the sample; and
determining a parameter of the sample, based on a relationship between the bulk-level measurand and the cellular-level measurand.

There is further provided, in accordance with some applications of the present invention, a method for use with a biological sample, the method including:
performing first and second optical measurements on the sample, using one or more optical measurement devices under respective sets of measuring conditions that are different from each other;
measuring a measurand of the sample, based upon the first optical measurement;
measuring a measurand of the sample, based upon the second optical measurement; and
based on a relationship between the measurand measured based upon the first optical measurement and the measurand measured based upon the second optical measurement, determining a relationship between the measuring conditions of the one or more optical measurement devices that were used to perform the first and second optical measurements.

In some applications, the biological sample includes a blood sample, and performing first and second optical measurements on the sample includes performing first and second optical measurements on the blood sample.

In some applications:
performing first and second optical measurements on a sample includes performing first and second optical measurements on respective portions of the sample that are disposed in respective portions of one or more sample chambers having respective dimensions; and determining the relationship between the measuring conditions of the one or more optical measurement devices that were used to perform the first and second optical measurements includes determining a relationship between dimensions of the respective portions of the one or more sample chambers.

In some applications, performing the first and second optical measurements on the sample includes performing at least one of the first and second optical measurements by acquiring an image of at least a portion of the sample, and determining the relationship between the measuring conditions of the one or more optical measurement devices that were used to perform the first and second optical measurements includes determining a field of view of the image.

In some applications, performing the first and second optical measurements on the sample includes performing at least one of the first and second optical measurements by acquiring an image of at least a portion of the sample, and determining the relationship between the measuring conditions of the one or more optical measurement devices that were used to perform the first and second optical measurements includes determining a level of magnification of the image.

In some applications:
measuring the measurand of the sample, based upon the first optical measurement includes measuring a given measurand of the sample, based upon the first optical measurement;
measuring the measurand of the sample, based upon the second optical measurement includes measuring the same given measurand of the sample, based upon the second optical measurement; and
determining the relationship between the measuring conditions of the one or more optical measurement devices that were used to perform the first and second optical measurements includes determining the relationship between the measuring conditions of the one or more optical measurement devices that were used to perform the first and second optical measurements, based upon a relationship the given measurand as measured based upon the first optical measurement, and the given measurand as measured based upon the second optical measurement.

In some applications, performing the first optical measurement includes performing the first optical measurement using a given optical measurement device, and performing the second optical measurement includes performing the second optical measurement using the same given optical measurement device.

In some applications, performing the first optical measurement includes performing the first optical measurement using a first optical measurement device, and performing the second optical measurement includes performing the second optical measurement using a second optical measurement device that is different from the first optical measurement device.

In some applications:
performing the first optical measurement includes performing the first optical measurement using a first optical measurement device that is configured to measure a parameter of one or more components within the sample, the parameter being selected from the group consisting of: optical absorption, transmittance, fluorescence, and luminescence; and
performing the second optical measurement includes performing the second optical measurement using a microscope configured to acquire a microscopic image of the sample.

In some applications:
measuring the measurand of the sample, based upon the first optical measurement includes measuring a first measurand of the sample, based upon the first optical measurement; and
measuring the measurand of the sample, based upon the second optical measurement includes measuring a second measurand of the sample that is different from the first measurand, based upon the second optical measurement; and
determining the relationship between the measuring conditions of the one or more optical measurement devices that were used to perform the first and second optical measurements includes determining the relationship between the measuring conditions of the one or more optical measurement devices that were used to perform the first and second optical measurements, based upon a relationship between the first and second measurands.

In some applications, measuring the first measurand includes measuring a bulk-level measurand of the sample, and measuring the second measurand includes measuring a cellular-level measurand of the sample.

There is further provided, in accordance with some applications of the present invention, apparatus for use with a biological sample, the apparatus including:
at least one computer processor configured to:
   perform first and second optical measurements on the sample, using one or more optical measurement devices under respective sets of measuring conditions that are different from each other,
   measure a measurand of the sample, based upon the first optical measurement,
   measure a measurand of the sample, based upon the second optical measurement, and
   based on a relationship between the measurand measured based upon the first optical measurement and the measurand measured based upon the second optical measurement, determine a relationship between the measuring conditions of the one or more optical measurement devices that were used to perform the first and second optical measurements.

There is further provided, in accordance with some applications of the present invention, a computer software product, for use with a biological sample, the computer software product including a non-transitory computer-readable medium in which program instructions are stored, which instructions, when read by a computer cause the computer to perform the steps of:
performing first and second optical measurements on a sample, using one or more optical measurement devices under respective sets of measuring conditions that are different from each other;
measuring a measurand of the sample, based upon the first optical measurement;
measuring a measurand of the sample, based upon the second optical measurement; and
based on a relationship between the measurand measured based upon the first optical measurement and the measurand measured based upon the second optical measurement, determining a relationship between the measuring conditions of the one or more optical measurement devices that were used to perform the first and second optical measurements.

There is further provided, in accordance with some applications of the present invention, a method for use with a biological sample, the method including:
performing first and second optical measurements on the sample, using one or more optical measurement devices under respective sets of measuring conditions that are different from each other;
measuring a measurand of the sample, based upon the first optical measurement;
measuring a measurand of the sample, based upon the second optical measurement;
normalizing the measurand measured based upon the first optical measurement and the measurand measured based upon the second optical measurement, with respect to each other; and
determining a parameter of the sample based upon at least one of the normalized measurand measured based upon the first optical measurement and the normalized measurand measured based upon the second optical measurement.

There is further provided, in accordance with some applications of the present invention, apparatus for use with a biological sample, the apparatus including:
at least one computer processor configured to:
   perform first and second optical measurements on the sample, using one or more optical measurement devices under respective sets of measuring conditions that are different from each other,
   measure a measurand of the sample, based upon the first optical measurement,
   measure a measurand of the sample, based upon the second optical measurement,
   normalize the measurand measured based upon the first optical measurement and the measurand measured based upon the second optical measurement, with respect to each other, and
   determine a parameter of the sample based upon at least one of the normalized measurand measured based upon the first optical measurement and the normalized measurand measured based upon the second optical measurement.

There is further provided, in accordance with some applications of the present invention, a computer software product, for use with a biological sample, the computer software product including a non-transitory computer-readable medium in which program instructions are stored, which instructions, when read by a computer cause the computer to perform the steps of:
performing first and second optical measurements on a sample, using one or more optical measurement devices under respective sets of measuring conditions that are different from each other;
measuring a measurand of the sample, based upon the first optical measurement;
measuring a measurand of the sample, based upon the second optical measurement;
normalizing the measurand measured based upon the first optical measurement and the measurand measured based upon the second optical measurement, with respect to each other; and
determining a parameter of the sample based upon at least one of the normalized measurand measured based upon the first optical measurement and the normalized measurand measured based upon the second optical measurement.

The present invention will be more fully understood from the following detailed description of embodiments thereof, taken together with the drawings, in which:

### BRIEF DESCRIPTION OF THE DRAWINGS

FIG. 1 is a block diagram showing components of a biological sample analysis system, in accordance some applications of the present invention;

FIG. 2 is a schematic illustration of a sample carrier, in accordance with some applications of the present invention;

FIG. 3 is a flowchart showing steps of algorithm that is performed, in accordance with some applications of the present invention;

FIG. 4 is a flowchart showing steps of algorithm that is performed, in accordance with some applications of the present invention;

FIG. 5 is a flowchart showing steps of algorithm that is performed, in accordance with some applications of the present invention;

FIG. 6 is a flowchart showing steps of algorithm that is performed, in accordance with some applications of the present invention;

FIG. 7 is a flowchart showing steps of algorithm that is performed in accordance with some applications of the present invention; and

FIG. 8 is a schematic cross-sectional illustration of a sample carrier that defines a variation in height that is stepped, in accordance with some applications of the present invention.

### DETAILED DESCRIPTION OF EMBODIMENTS

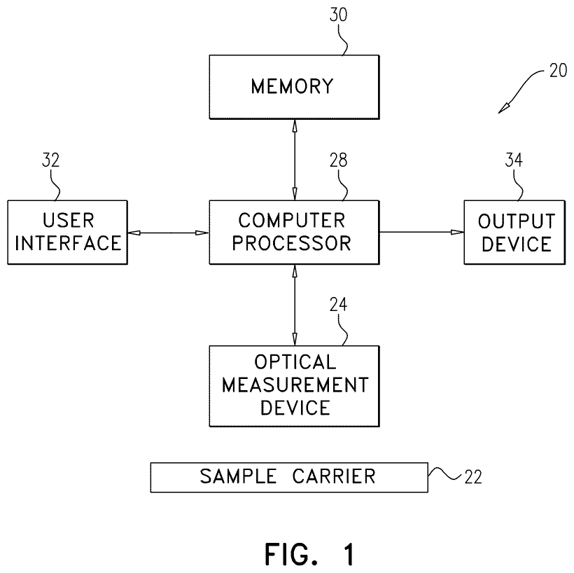

Reference is now made to FIG. 1, which is a block diagram showing components of a biological sample analysis system 20, in accordance with some applications of the present invention. Typically, a biological sample (e.g., a blood sample) is placed into a sample carrier 22. While the sample is disposed in the sample carrier, optical measurements are performed upon the sample using one or more optical measurement devices 24. For example, the optical measurement devices may include a microscope (e.g., a digital microscope), a spectrophotometer, a photometer, a spectrometer, a camera, a spectral camera, a hyperspectral camera, a fluorometer, a spectrofluorometer, and/or a photodetector (such as a photodiode, a photoresistor, and/or a phototransistor). For some applications, the optical measurement devices include dedicated light sources (such as light emitting diodes, incandescent light sources, etc.) and/or optical elements for manipulating light collection and/or light emission (such as lenses, diffusers, filters, etc.). For some applications, a microscope system is used that is generally similar to the microscope system described in US 2014/0347459 to Greenfield, which is incorporated herein by reference.

A computer processor 28 typically receives and processes optical measurements that are performed by the optical measurement device. Further typically, the computer processor controls the acquisition of optical measurements that are performed by the one or more optical measurement devices. The computer processor communicates with a memory 30. A user (e.g., a laboratory technician) sends instructions to the computer processor via a user interface 32. For some applications, the user interface includes a keyboard, a mouse, a joystick, a touchscreen device (such as a smartphone or a tablet computer), a touchpad, a trackball, a voice-command interface, and/or other types of user interfaces that are known in the art. Typically, the computer processor generates an output via an output device 34. Further typically, the output device includes a display, such as a monitor, and the output includes an output that is displayed on the display. For some applications, the processor generates an output on a different type of visual, text, graphics, tactile, audio, and/or video output device, e.g., speakers, headphones, a smartphone, or a tablet computer. For some applications, user interface 32 acts as both an input interface and an output interface, i.e., it acts as an input/output interface. For some applications, the processor generates an output on a computer-readable medium (e.g., a non-transitory computer-readable medium), such as a disk, or a portable USB drive, and/or generates an output on a printer.

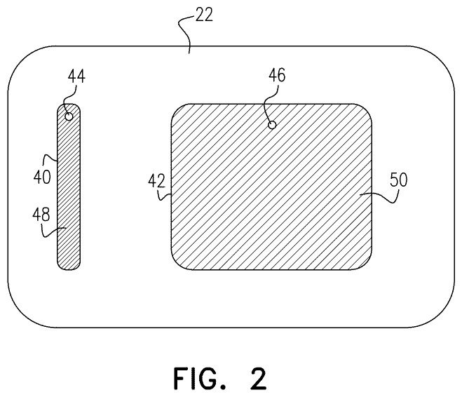

Reference is now made to FIG. 2, which is a schematic illustration of sample carrier 22, in accordance with some applications of the present invention. For some applications, the sample carrier includes a source sample portion chamber 40, as well as a diluted sample portion chamber 42. Typically, chambers 40 and 42 are filled via respective entry holes 44 and 46.

For some applications, diluted sample portion chamber 42 is filled with a second portion 50 of a biological sample (e.g., a portion of a blood sample), which is diluted with respect to a first portion 48 of the sample that is placed in the source sample portion chamber 40. For example, a portion of the sample may be diluted in order to identify and/or count components of the sample, which may be less easily identified and/or counted in an undiluted portion of the sample. For some applications, the diluted portion includes a staining substance. For example, a diluted portion may be prepared using techniques as described in US 2015/0316477 to Pollak, which is incorporated herein by reference, and which describes a method for preparation of blood samples for analysis that involves a dilution step, the dilution step facilitating the identification and/or counting of components within microscopic images of the sample. Typically, in such applications, although the extent of dilution is typically set as part of the protocol, small variations in the dilution can lead to corresponding errors in the absolute quantification of the different components and/or analytes within the sample. In accordance with some applications of the present invention, two different measurements are performed upon, respectively, first portion 48 of the sample that is placed in chamber 40 (i.e., a source sample portion), and second portion 50 of the sample that is placed in chamber 42, and which is diluted with respect to the first portion (i.e., a diluted sample portion). For some applications, based upon the measurements, the dilution factor (i.e., the dilution ratio, and/or the extent to which the second portion is diluted with respect to the first portion) is determined. Typically, a normalization factor is determined, the normalization factor being a property of the source sample portion to which other measurements are correlated (e.g. the number of red blood cells per unit area or per unit volume in the source sample portion). Further typically, measurands within the sample (e.g., within the source sample portion) are measured based upon the normalization factor, as described in further detail hereinbelow.

For some applications, the methods described herein are performed with respect to a source sample portion and a diluted sample portion without the portions being placed into respective chambers of a single sample carrier, as shown in FIG. 2. For some applications, the methods described herein are performed with respect to first and second portions of a sample, which are not diluted with respect to one another, mutatis mutandis. For some such applications, respective measurements (e.g., respective optical measurements) that are performed upon the first and second portions are normalized with respect to one another, using techniques as described herein.

For some applications, source sample portion 48, which is placed in the source sample portion chamber is a natural undiluted biological fluid (e.g., a blood sample or urine sample), or is a sample that underwent some modification, including, for example, one or more of dilution (e.g., dilution in a controlled fashion), addition of a component or reagent, or fractionation. Diluted sample portion 50, which is placed within the diluted sample portion chamber, is typically diluted with respect to the portion of the sample that is placed in the source sample portion chamber. For example, the diluent may contain pH buffers, stains, fluorescent stains, antibodies, sphering agents, lysing agents, etc.

Typically, an assay is performed on diluted sample portion 50 that provides a plurality of measurements, which are assumed to have good relative agreement with each other. Further typically, an assay is performed on source sample portion 48 that yields a measurement that corresponds to at least one of the measurements performed upon the diluted sample portion. For some applications, at least one of the measurements performed on diluted sample portion 50 is normalized by normalizing using measurements that are measured and/or derived from both portions 48 and 50.

For example, a blood sample may be diluted using a dilution technique as described in US 2015/0316477 to Pollak, which is incorporated herein by reference, and the smear may be suitably stained and imaged using a microscope system (which may be manual or automated). For some applications, the microscope system is one of optical measurement devices 24, described hereinabove with respect to FIG. 1. For some applications, the microscope images are analyzed (e.g. manually, or using a computer processor that runs suitable computer software) to identify different blood cells. However, an error of 10 percent in the dilution factor may correspond directly to a 10 percent error in the count of, for example, red blood cell per unit volume (e.g., per microliter) of blood. Such errors in dilution can originate from a number of sources. Illustrative examples of sources of such errors (which are not intended to limit the scope of the present invention) include pipetting inaccuracy or error, calibration inaccuracy or error, mixing inaccuracy or error, etc.

For some applications, the methods described herein are used to account for at least some of the variation in dilution by making a measurement on a source sample portion (e.g., an undiluted blood sample portion) from which the diluted sample portion is extracted. This measurement typically corresponds to at least one of the measurements measured on the diluted sample portion. For example, the measurement performed on the source sample portion may include measurement of: hemoglobin content, white blood cell content, red blood cell content, hematocrit, content of a specific white blood cell type, platelet content, and/or any measurand that is measured or that can be inferred for the diluted sample portion. For some applications, a normalization factor is determined, the normalization factor being a property of the source sample portion to which other measurements are correlated (e.g. the number of red blood cells per unit area or per unit volume in the source sample portion). Typically, measurands within the sample (e.g., within the source sample portion) are measured based upon the normalization factor, as described in further detail hereinbelow. For some applications, a plurality of measurements (for example, two or more of the above-described measurements) are performed on source sample portion 48, and a normalization factor (e.g., a dilution factor) is determined based upon the plurality of measurements. Typically, in such cases, a normalization factor is determined based upon the different components of data, using a statistical method (e.g. averaging, regression, curve-fitting or other techniques known in the art). For some applications, the accuracy of the normalization is increased by using a plurality of analytical measurements in the above-described manner, relative to if only a single measurement is used.

Typically, as described hereinabove, the above-described method is performed on two or more portions of the sample that are at different levels of dilution (i.e., a source sample portion and a dilution sample portion), whereby the amount or concentration of different components in one sample portion is determined based on a dilution factor between the two sample portions. For example, the method may be used to determine the amount or concentration of different blood components in a complete blood count assay that is conducted on a diluted blood sample portion.

For some applications, a dilution factor (i.e., the dilution ratio (such as 1:100), and/or the extent to which the second portion is diluted with respect to the first portion) is determined. For some applications, the same measurand is measured in the diluted and source sample portions. For example, a count per unit volume of a component, a concentration of a component, and/or an optical density of a component may be measured. The dilution factor for the diluted sample portion relative to the source sample portion is derived from the ratio of the measurand as measured within the two sample portions (e.g., the ratio of the count per unit volume of the component, the concentration of the component, and/or the optical density of the component as measured within the two sample portions). The dilution factor is typically used to determine a parameter relating to (e.g., the count per unit volume, the concentration, and/or the optical density of) one or more other components.

As described hereinabove, typically, a normalization factor is determined that is a property of the source sample portion to which other measurements are correlated (e.g., the number of red blood cells per unit area or per unit volume in the source sample portion). For some applications, one measurand is measured in the undiluted sample portion and a different measurand is measured in the diluted sample portion. For example, hemoglobin concentration (Hb) may be measured in a source blood sample portion, and mean corpuscular hemoglobin (MCH) may be measured in a diluted blood sample portion. Or, the hematocrit may be measured in a source blood sample portion, and mean corpuscular volume (MCV) may be measured in a diluted blood sample portion. Typically, a relationship between the two measurements is determined (e.g., the two measurements may be divided by one another), and the concentration, or count per unit volume, of a reference component in the source sample portion is inferred based upon the relationship. Thereafter, a parameter relating to (e.g., the count per unit volume, the concentration, and/or the optical density of) one or more other components is determined in correlation to a ratio of the other component with respect to the reference component. The above-described techniques will be more easily understood by means of the following examples.

For some applications (e.g., in the context of a complete blood count), a measurement is performed upon an undiluted sample to determine the total hemoglobin concentration (“Hb”), for example, using optical density measurements conducted on undiluted blood. Typically, such measurements are performed using a spectrophotometer, spectrometer, camera, a spectral camera, a hyperspectral camera, as optical measurement device 24 (FIG. 1). The mean corpuscular hemoglobin (MCH) is determined using a diluted blood sample portion. For example, optical density measurements may be performed on a cellular level (i.e., with respect to individual cells). For example, a microscope may be used as an optical measurement device 24 (FIG. 1), and cells may be imaged using bright-field imaging under violet or green wavelengths. For some applications, the red blood cell count per unit volume (“RBC”) in the source sample portion is deduced by dividing the hemoglobin concentration by the mean corpuscular hemoglobin (since RBC=Hb/MCH). For some applications, based upon the red blood cell count per unit volume in the source sample portion, the count of additional components within the source sample portion is determined. For example, using a microscopic image of the diluted sample portion, the ratio of counts of other blood components (e.g. red blood cells of a given type, white blood cells, white blood cells of a given type, circulating tumor cells, platelets, platelets of a given type, bacteria, pathogens, pathogens of a given type, reticulocytes, and Howell-Jolly bodies) to the red blood cell count may be determined. Alternatively or additionally, the ratio of counts of other blood components to the red blood cell count may be determined using a microscopic image of a non-diluted sample portion, which forms a monolayer having a sufficiently low cell density for identifying individual components within the monolayer (for example, by virtue of the portion having been placed in a sample chamber having a relatively low height). The absolute count of the additional components within the source sample portion is determined by multiplying this ratio by the red blood cell count. For example, once the white blood cell to red blood cell ratio is determined in the diluted sample portion as (WBC/RBC)_{diluted}, the white blood cell count per unit volume in the source sample portion (“WBC_{count}”) is calculated as WBC_{count}=(WBC/RBC)_{diluted}×RBC=(WBC/RBC)_{diluted}×Hb/MCH. For some applications, the hematocrit within the sample is determined based upon the determined red blood cell count. For example, the mean corpuscular volume may be measured with respect to the diluted sample portion, and the hematocrit may be determined by multiplying the mean corpuscular volume by the red blood cell count.

For some applications (e.g., in the context of a complete blood count), a measurement is performed upon an undiluted sample to determine the hematocrit (“HCT”), for example, using the micro-hematocrit method (in which a volume of blood is centrifuged), or using ultrasonic and/or impedance measurements. The mean corpuscular volume (“MCV”) is determined using a diluted blood sample portion. For example, the red blood cells may be imaged using a microscope as optical measurement device 24 (FIG. 1), and the mean corpuscular volume may be derived from the images. For some applications, the red blood cell count per unit volume (“RBC”) in the source sample portion is deduced by dividing the hematocrit by the mean corpuscular volume (since RBC=HCT/MCV). For some applications, based upon the red blood cell count per unit volume in the source sample portion, the count of additional components within the source sample portion is determined. For example, using a microscopic image of the diluted sample portion, the ratio of counts of other blood components (e.g. red blood cells of a given type, white blood cells, white blood cells of a given type, circulating tumor cells, platelets, platelets of a given type, bacteria, pathogens, pathogens of a given type, reticulocytes, and Howell-Jolly bodies) to the red blood cell count may be determined. Alternatively or additionally, the ratio of counts of other blood components to the red blood cell count may be determined using a microscopic image of a non-diluted sample portion, which forms a monolayer having a sufficiently low cell density for identifying individual components within the monolayer (for example, by virtue of the portion having been placed in a sample chamber having a relatively low height). The absolute count of the additional components within the source sample portion is determined by multiplying this ratio by the red blood cell count. For example, once the white blood cell to red blood cell ratio is determined in the diluted sample as (WBC/RBC)_{diluted}, the white blood cell count per unit volume in the source sample portion (“WBC_{count}”) is calculated as WBC_{count}=(WBC/RBC)_{diluted}×RBC=(WBC/RBC)_{diluted}×HCT/MCV. For some applications, hemoglobin concentration within the sample is determined based upon the determined red blood cell count. For example, the mean corpuscular hemoglobin may be measured with respect to the diluted sample portion, and the hemoglobin concentration may be determined by multiplying the mean corpuscular hemoglobin by the red blood cell count.

For some applications (e.g., in the context of a complete blood count), the total white blood cell count per unit volume is determined in the source sample portion. For example, the total white blood cell count per unit volume may be determined by (a) lysing the red blood cells within the sample (such that the red blood cells don't cause scattering of light), (b) imaging the sample using a DNA-specific stain such as Methylene Blue, which has a high absorption in wavelengths in which the absorbance of hemoglobin from the red blood cells is low, and (c) measuring light absorption at those wavelengths. For some applications, based upon the white blood cell count per unit volume in the source sample portion, the count of additional components within the source sample portion is determined. For example, using a microscopic image of the diluted sample portion, the ratio of counts of other blood components (e.g. red blood cells, red blood cells of a given type, white blood cells of a given type, circulating tumor cells, platelets, platelets of a given type, bacteria, pathogens, pathogens of a given type, reticulocytes, and Howell-Jolly bodies) to the white blood cell count may be determined. The absolute count of the additional components within the source sample portion is determined by multiplying this ratio by the white blood cell count.

For some applications, even source sample portion 48 is not a natural biological sample, but has itself been diluted, for example. For some such applications, the counts and/or concentrations of components within the natural sample, from which the source sample portion was produced, are derived. For example, natural blood may be diluted in a controlled, precise matter to produce a source sample portion, with the dilution factor of this dilution step being precisely known. The source sample portion is then used as described hereinabove to produce diluted sample portion 50, and the count per unit volume and/or concentration of some blood components in the source sample portion is derived, as described hereinabove. Based upon the count per unit volume and/or concentration of blood components in the source sample portion, the count per unit volume and/or concentration of those components within the natural sample are derived.

For some applications, a natural sample is diluted to produce source sample portion 48, which is diluted further to produce diluted sample portion 50. Based on parameters determined for each of the source sample portion and the diluted sample portion, parameters are extrapolated for the natural sample, without directly estimating a dilution factor. For example, the ratio of white blood cells to red blood cells may be determined using microscopic images of the diluted sample portions, as described hereinabove, while the ratio of basophils to white blood cells may be determined for the source sample portion. In addition, the red blood cell count per unit volume may be determined for the natural sample. The basophils count per unit volume of the natural sample may thereby be determined, using the red blood cell count per unit volume for the natural sample in combination with the ratios.

Referring again to FIG. 2, for some applications, the techniques described herein are performed using carrier 22, the carrier having at least two chambers for each patient (or source), as described hereinabove. Typically, source sample portion chamber 40 is configured to assay a small volume, such as between 1 microliter and 30 microliters of blood, so as not to necessitate, for example, drawing much blood. Further typically, the source sample portion chamber and diluted sample portion chamber are in close proximity to one another, for example by being disposed upon a single sample carrier, as shown in FIG. 2. For some applications, the proximity of the source and diluted sample chambers to one another is beneficial in reducing the hazard of mismatching a source sample and a diluted sample.

As described hereinabove, for some applications, the methods described herein are performed with respect to a source sample portion and a diluted sample portion without the portions being placed into respective chambers of a single sample carrier, as shown in FIG. 2. For some applications, the methods described herein are performed with respect to first and second portions of a sample, which are not diluted with respect to one another, mutatis mutandis. For such applications, respective measurements (e.g., respective optical measurements) that are performed upon the first and second portions are normalized with respect to one another, using techniques as described herein.

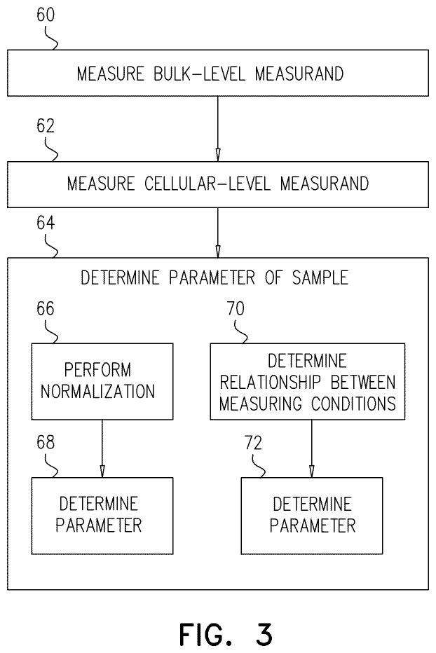

Although some of the above examples are described with reference to performing certain measurements with respect to source and diluted sample portions of a blood sample, the scope of the present invention includes generally performing combinations of measurements (e.g., optical measurements) on a sample (and/or portions thereof), to thereby derive a parameter of the sample, as described with reference to the flowcharts shown in FIGS. 3-7.

Reference is now made to FIG. 3, which is a flowchart showing steps of an algorithm that is performed, in accordance with some applications of the present invention. In accordance with some applications of the present invention, two or more measurements (which are typically optical measurements) are performed upon a biological sample. Typically, the biological sample is a blood sample. For some applications, a bulk-level measurand of the sample is measured, by performing a first measurement on the sample, in a first step 60. Further typically, a cellular-level measurand of the sample is measured, by performing a second measurement on the sample, in a second step 62. For the purpose of the present applications, the term “cellular-level measurand” should be understood to mean a measurand that relates to one or more parameters of individual cells or other non-dissolved components within the sample, such as mean corpuscular volume, mean corpuscular hemoglobin, mean platelet volume, and/or red blood cell distribution width, etc. Measurement of a cellular-level measurand typically involves a first step of identifying individual cells or other non-dissolved components within the sample (e.g., identifying such components within a microscopic image), and a second step of identifying a parameter of such individual identified components. For some applications, the cellular-level measurand is measured by analyzing one or more microscopic images of the sample. For the purpose of the present applications, the term “bulk-level measurand” should be understood to mean a measurand that relates a parameter of the sample as a whole, and that does not require the two steps of identifying individual cells or other non-dissolved components within the sample, and identifying a parameter of such individual identified components. For example, such a measurand may include the optical density of a given component (which is measured by performing a measurement on a bulk volume of the sample, e.g., even after performing lysis of individual components within the bulk volume), a count per unit volume of a given component (which is typically measured by identifying such components, but does not require identifying a parameter of individual identified components), and/or the concentration of a given component (such as red blood cell concentration, hemoglobin concentration, white blood cell concentration, platelet concentration, and/or hematocrit, i.e., red blood cell concentration). Typically, bulk-level measurands are measured by performing a measurement on a bulk volume of the sample. For example, such measurements may include ultrasonic, impedance, optical absorption, transmittance, fluorescence, microscopic and/or luminescence measurements that are performed on a bulk volume of the sample. In accordance with respective applications, the first and second measurements are performed on the same portion of the sample, or on respective, different portions of the sample.

Typically, in a third step 64, a parameter of the sample is determined, based on a relationship between the bulk-level measurand and the cellular-level measurand. For some applications, in a sub-step 66 of step 64, the first measurement is normalized with respect to the second measurement. Typically, a relationship between the two measurements is determined (e.g., the two measurements may be divided by one another), and the concentration, or count per unit volume, of a reference component is inferred based upon the relationship, as described hereinabove. For some applications, the second measurement is performed on a second portion of the sample that is diluted with respect to a first portion of the sample upon which the first measurement is performed, and in sub-step 66, a dilution ratio by which the second portion of the sample is diluted with respect to the first portion of the sample is determined. For some applications, in a further sub-step 68 of step 64, the parameter of the sample is determined, based upon the normalization, and a further measurement that is performed on the sample, as described in further detail herein.

For some applications, the first measurement is performed using a first set of measuring conditions, and the second measurement is performed using a second set of measuring conditions. For some such applications, in a sub-step 70 of step 64, a relationship between the sets of measuring conditions is determined. Typically, in a further sub-step 72, a parameter of the sample is determined based upon the relationship between the sets of measuring conditions. For example, the first and second optical measurements may be performed on respective portions of the sample that are disposed in respective portions of one or more sample chambers having respective dimensions (e.g., respective heights). For some such applications, a relationship between dimensions of the respective portions of the one or more sample chambers is determined, based on the relationship between the bulk-level measurand and the cellular-level measurand. Alternatively or additionally, a field of view from which one of the first and second optical measurements was measured (e.g., a microscopic image was acquired) is determined, and/or a level of magnification at which one of the first and second optical measurements was measured (e.g., a microscopic image was acquired) is determined. For some applications, the bulk-level measurand and the cellular-level measurand are normalized with respect to one another. Subsequently, a parameter of the sample is determined based upon the normalization of the bulk-level measurand and the cellular-level measurand with respect to one another.

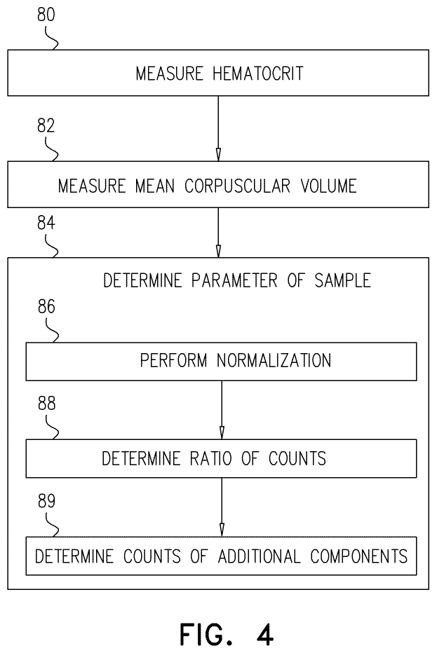

Reference is now made to FIG. 4, which is a flowchart showing steps of an algorithm that is performed, in accordance with some applications of the present invention. For some applications, in a first step 80, hematocrit is measured by performing a first measurement on a blood sample, and, in a second step 82, mean corpuscular volume within the blood sample is measured, by performing a second measurement on the blood sample. For example, hematocrit may be measured using the micro-hematocrit method, or using ultrasonic and/or impedance measurements, and the mean corpuscular volume may be measured by analyzing microscopic images that are acquired of a second portion of the blood sample. Typically, the second portion of the sample is diluted with respect to the first portion of the blood sample.

Typically, in a third step 84, a parameter of the sample is determined based upon the relationship between the hematocrit and the mean corpuscular volume. For some applications, in a sub-step 86 of step 84, based on the relationship between the hematocrit and the mean corpuscular volume, the first portion of the sample and second portion of the sample are normalized with respect to each other. Typically, the red blood cell count (e.g., count per unit volume) within the sample is determined by dividing the hematocrit by the mean corpuscular volume, such that the red blood cell count can thereby act as a reference parameter with reference to which other parameters are normalized. For some applications, counts of one or more additional components within the sample (e.g., red blood cells of a given type, white blood cells, white blood cells of a given type, circulating tumor cells, platelets, platelets of a given type, bacteria, pathogens, pathogens of a given type, reticulocytes, and/or Howell-Jolly bodies) are determined, based on the red blood cell count within the sample. For example, in a sub-step 88 of step 84, a ratio between the red blood cell count and the counts of the one or more additional components within a portion of the sample may be determined, by analyzing a microscopic image of the diluted portion of the sample. Subsequently, in a sub-step 89 of step 84, the counts of the one or more additional components are determined, based on the red blood cell count within the source sample portion and the ratio between the red blood cell count and the counts of the one or more additional components within the diluted portion of the sample.

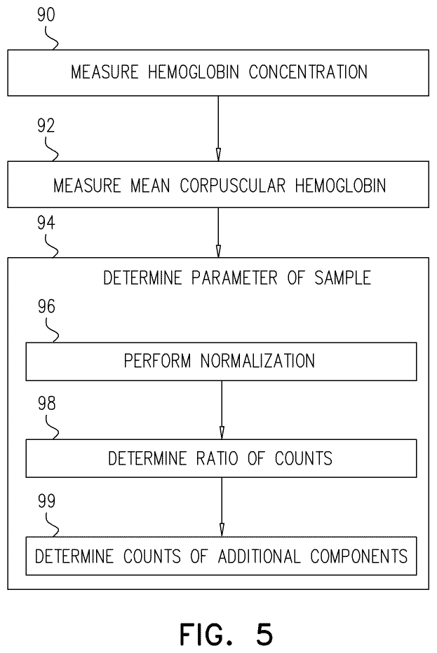

Reference is now made to FIG. 5, which is a flowchart showing steps of algorithm that is performed, in accordance with some applications of the present invention. For some applications, in a first step 90, hemoglobin concentration is measured by performing a first measurement on a blood sample, and, in a second step 92, mean corpuscular hemoglobin within the blood sample is measured, by performing a second measurement on the blood sample. For example, hemoglobin concentration may be measured by performing optical density measurements on a first portion of the blood sample, and the mean corpuscular hemoglobin may be measured by performing optical density measurements on a cellular level (i.e., with respect to individual cells) on a second portion of the sample. Typically, the second portion of the sample is diluted with respect to the first portion of the blood sample.

Typically, in a third step 94, a parameter of the sample is determined based upon the relationship between the hemoglobin concentration and the mean corpuscular hemoglobin. For some applications, in a sub-step 96 of step 94, based on the relationship between the hemoglobin concentration and the mean corpuscular hemoglobin, the first portion of the sample and second portion of the sample are normalized with respect to each other. Typically, the red blood cell count (e.g., count per unit volume) within the sample is determined by dividing the hemoglobin concentration and the mean corpuscular hemoglobin, such that the red blood cell count can thereby act as a reference parameter with reference to which other parameters are normalized. For some applications, counts of one or more additional components within the sample (e.g., red blood cells of a given type, white blood cells, white blood cells of a given type, circulating tumor cells, platelets, platelets of a given type, bacteria, pathogens, pathogens of a given type, reticulocytes, and/or Howell-Jolly bodies) are determined, based on the red blood cell count within the sample. For example, in a sub-step 98 of step 94, a ratio between the red blood cell count and the counts of the one or more additional components within the diluted portion of the sample may be determined, by analyzing a microscopic image of the portion of the diluted portion of the sample. Subsequently, in a sub-step 99 of step 94, the counts of the one or more additional components are determined, based on the red blood cell count within the source sample portion and the ratio between the red blood cell count and the counts of the one or more additional components within the diluted portion of the sample.

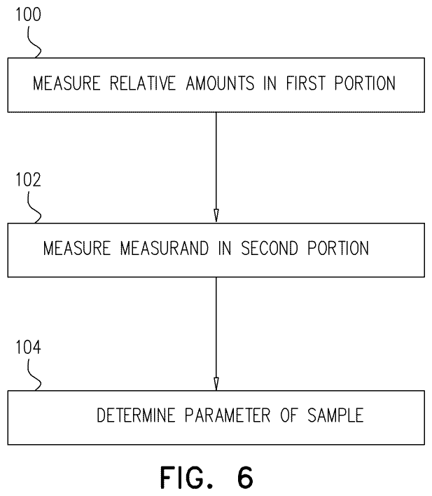

Reference is now made to FIG. 6, which is a flowchart showing steps of an algorithm that is performed, in accordance with some applications of the present invention. For some applications, in a first step 100, relative amounts of first and second components are measured within the first portion of the blood sample. In a second step 102, a measurand is measured within a second portion of the blood sample. In a third step 104, a parameter of the blood sample is determined based upon a relationship between the relative amounts of first and second components within the first portion of the blood sample, and the measurand within the second portion of the blood sample. For some applications, the steps described in the flowchart shown in FIG. 6 are performed in combination with steps shown in any one of the other flowcharts.

Typically, step 100 is performed by analyzing a microscopic image of the first portion of the blood sample. For some applications, the first portion is diluted with respect to the second portion, e.g., as described hereinabove. (It is noted that the diluted and source portions of the sample are described interchangeably as first and second portions of the sample.)

For some applications, in step 100, relative amounts of all white blood cell types, neutrophils, eosinophils, basophils, lymphocytes, monocytes, and/or white blood cell precursors are measured, e.g., by analyzing a microscopic image of the first portion of the blood sample. In step 102, the absolute count of all types of white blood cells is determined. For some applications, step 102 is performed by performing a bulk-level measurement, e.g., by performing an optical density measurement upon a source sample portion. In step 104, the absolute counts of respective types of white blood cells (or of a given type of white blood cell) is determined, based upon steps 100 and 102.

For some applications, in step 100, relative amounts of red blood cells, reticulocytes, intracellular bodies, red blood cells having a given morphology, and/or Howell-Jolly bodies are measured, e.g., by analyzing a microscopic image of the first portion of the blood sample. In step 102, the absolute count of all types of the above-described components is determined, e.g., by performing an optical density measurement upon a source sample portion. In step 104, the absolute counts of respective types of the above-described components (or of a given one of the above-described components) is determined, based upon steps 100 and 102.

For some applications, in step 100, relative amounts of reticulocyted platelets, giant platelets, and/or regular platelets are measured, e.g., by analyzing a microscopic image of the first portion of the blood sample. In step 102, the absolute count of all platelet types is determined, e.g., by performing an optical density measurement upon a source sample portion. In step 104, the absolute counts of respective types of platelets (or of a given type of platelet) is determined, based upon steps 100 and 102.

For some applications, combinations of different cell types are analyzed using the technique described with reference to FIG. 6. For example, ratios of any combination of red blood cells, red blood cells of given types, white blood cells, white blood cells of given types, platelets, platelets of given types, intracellular bodies, precursor cells, circulating tumor cells, pathogens, pathogens of a given type, reticulocytes, and/or Howell-Jolly bodies, etc. may be measured in the first portion, and in the second portion absolute counts of any of the aforementioned components may be measured, such as to derive an absolute count of another one of the components, mutatis mutandis.

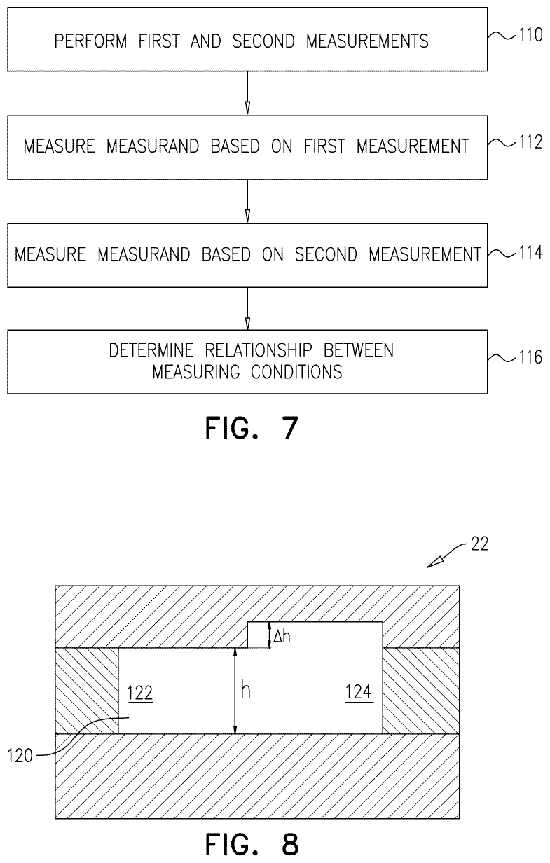

Reference is now made to FIG. 7, which is a flowchart showing steps of an algorithm that is performed, in accordance with some applications of the present invention. For some applications, in a first step 110, first and second optical measurements are performed on a sample, using one or more optical measurement devices under respective sets of measuring conditions that are different from each other. Typically, in a second step 112, a measurand of the sample is measured, based upon the first optical measurement, and, in a third step 114, a measurand of the sample is measured, based upon the second optical measurement. In accordance with respective applications, the measurand that is measured based upon the second optical measurement is the same as the measurand that is measured based upon the first optical measurement, or is different from the measurand that is measured based upon the first optical measurement. In accordance with respective applications, the first and second optical measurements are performed on the same portion of the sample, or on different portions of the sample. For some applications, one of the optical measurements is performed on a portion of the sample that is diluted with respect to a portion of the sample upon which the other optical measurement is performed.

Typically, in a fourth step 116, based on a relationship between the measurand measured based upon the first optical measurement and the measurand measured based upon the second optical measurement, a relationship between the measuring conditions of the one or more optical measurement devices that were used to perform the first and second optical measurements is determined. For example, a field of view from which one of the first and second optical measurements was measured (e.g., a microscopic image was acquired) is determined, and/or a level of magnification at which one of the first and second optical measurements was measured (e.g., a microscopic image was acquired) is determined. For some applications, the first and second optical measurements are normalized with respect to one another, and a parameter of the sample is determined based upon the normalized measurements, e.g., using techniques described herein.

For some applications, the first measurement is performed using a first type of optical measurement device (e.g., a device configured to perform cellular-level measurements, such as a microscope), and the second measurement is performed using a second type of optical measurement device (e.g., a device configured to perform bulk-level measurements, such as a spectrophotometer, a photometer, a spectrometer, a camera, a spectral camera, a hyperspectral camera, a fluorometer, a spectrofluorometer, and/or a photodetector). Measurements using the respective types of devices are normalized with respect to each other, such as to account for errors and/or inaccuracies in one or both of the devices. For example, the normalization may account for errors in the level of magnification of a microscope, and/or the gain of a device configured to perform bulk-level measurements, such as a spectrophotometer, a photometer, a spectrometer, a camera, a spectral camera, a hyperspectral camera, a fluorometer, a spectrofluorometer, and/or a photodetector.

Reference is now made to FIG. 8, which is a schematic cross-sectional illustration of sample carrier 22, in accordance with some applications of the present invention. For some applications, the sample carrier defines one or more sample chambers 120, into which the sample is placed, and the one or more sample chambers define at least a first region 122 (which is shallower) and a second region 124 (which is deeper), the height of the one or more sample chambers varying between the first and second regions. (For example, as shown, the height of the first region is h, and the height of the second region is (h+Δh).) For some applications, a first optical measurement is performed on a first portion of the sample, which is disposed within the first region, and a second optical measurement is performed on a second portion of the sample, which is disposed in the second region. For example, such measurements may be performed in accordance with techniques described in an International application being filed on even date herewith, entitled “Sample carrier for optical measurements,” which is incorporated herein by reference. For some such applications, a technique as described with respect to FIG. 7 is performed, in which, in step 116, the relationship between the heights of the respective portions of the one or more sample chambers is determined, based on the relationship between the measurand measured based upon the first optical measurement and the measurand measured based upon the second optical measurement.

It is noted with reference to the flowcharts shown in FIGS. 3-7 that the steps of the flowchart are not necessarily performed in the order in which they appear in the flowcharts. For some applications, steps of the flowcharts shown in FIGS. 3-7 are performed in combination with one another. It is further noted that, in general, in response to the parameters of the sample that are determined using the techniques described herein, an output is generated, e.g., via user interface 32, and/or output device 34, both of which are shown in FIG. 1.

For some applications, the sample as described herein is a sample that includes blood or components thereof (e.g., a diluted or non-diluted whole blood sample, a sample including predominantly red blood cells, or a diluted sample including predominantly red blood cells), and parameters are determined relating to components in the blood such as platelets, white blood cells, anomalous white blood cells, circulating tumor cells, red blood cells, reticulocytes, Howell-Jolly bodies, etc.

Although some applications of the present invention have been described with reference to performing a complete blood count, and/or with respect to the analysis of blood in general, the scope of the present invention includes using the techniques described herein to perform other types of analysis, mutatis mutandis. For example, the techniques described herein may be applied to methods related to quantifying blood cells and/or other analytes in blood, methods for analyzing urine (e.g. for cell clumps), cerebral-spinal fluid (CSF), gynecological samples, fecal samples, synovial fluid samples, saliva, semen, sweat, sputum, vaginal fluid, breast milk, bronchoalveolar lavage, gastric lavage, tears, nasal discharge, biological excretions or other biological samples originating from humans or other species. The techniques are not limited to the counting of cells and can be used for the quantification of other analytes such as proteins, peptides, small molecules, infectious agents, etc. The biological sample may be from any living creature, and is typically from warm blooded animals. For some applications, the biological sample is a sample from a mammal, e.g., from a human body. For some applications, the sample is taken from any domestic animal, zoo animals and farm animals, including but not limited to dogs, cats, horses, cows and sheep. Alternatively or additionally, the biological sample is taken from animals that act as disease vectors including deer or rats.

For some applications, similar techniques to those described hereinabove are applied to a non-bodily sample. For some applications, the sample is an environmental sample, such as, a water (e.g. groundwater) sample, surface swab, soil sample, air sample, or any combination thereof. In some embodiments, the sample is a food sample, such as, a meat sample, dairy sample, water sample, wash-liquid sample, beverage sample, and any combination thereof. For some applications, the techniques described herein are applied to the analysis of non-biological substances, such as the analysis of analytes in an industrial setting.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

A data processing system suitable for storing and/or executing program code will include at least one processor (e.g., computer processor 28) coupled directly or indirectly to memory elements (e.g., memory 30) through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that blocks of the flowcharts shown in FIGS. 3, 4, 5, 6, and 7, and combinations of blocks in the flowcharts, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the flowcharts and/or algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the flowchart blocks and algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the flowcharts and/or algorithms described in the present application.

Computer processor 28 is typically a hardware device programmed with computer program instructions to produce a special purpose computer. For example, when programmed to perform the algorithms described with reference to FIGS. 3, 4, 5, 6, and 7, computer processor 28 typically acts as a special purpose sample-analysis computer processor. Typically, the operations described herein that are performed by computer processor 28 transform the physical state of memory 30, which is a real physical article, to have a different magnetic polarity, electrical charge, or the like depending on the technology of the memory that is used.

The apparatus and methods described herein may be used in conjunction with apparatus and methods described in any one of the following patent applications, all of which are incorporated herein by reference:
US 2012/0169863 to Bachelet;
US 2014/0347459 to Greenfield;
US 2015/0037806 to Pollak;
US20150316477 to Pollak;
US20160208306 to Pollak;
US20160246046 to Yorav Raphael;
US20160279633 to Bachelet;
WO 16/030897 to Yorav Raphael;
WO 17/046799 to Eshel; and
WO 17/168411 to Eshel.

There is provided, in accordance with some applications of the present invention, the following inventive concepts:
1. A method for use with a biological sample, the method comprising:
   measuring a bulk-level measurand of the sample, by performing a first measurement on the sample;
   measuring a cellular-level measurand of the sample, by performing a second measurement on the sample; and
   determining a parameter of the sample, based on a relationship between the bulk-level measurand and the cellular-level measurand.
2. The method according to inventive concept 1, wherein determining the parameter of the blood sample comprises normalizing the first and second measurements with respect to each other, based on the relationship between the bulk-level measurand and the cellular-level measurand.
3. The method according to inventive concept 1, wherein measuring the bulk-level measurand comprises determining an optical density of a given component within the sample.
4. The method according to inventive concept 1, wherein measuring the cellular-level measurand comprises analyzing a microscopic image of the sample.
5. The method according to inventive concept 1, wherein performing the first measurement on the sample comprises performing the first measurement on the sample using a first set of measuring conditions, wherein performing the second measurement on the sample comprises performing the second measurement on the sample using a second set of measuring conditions, and wherein determining the parameter of the sample comprises determining a relationship between the measuring conditions that were used to perform the first and second measurements, based on the relationship between the bulk-level measurand and the cellular-level measurand.
6. The method according to inventive concept 1, wherein performing the first measurement comprises performing the first measurement on a first portion of the sample, and wherein performing the second measurement comprises performing the second measurement upon the first portion of the sample.
7. The method according to any one of inventive concepts 1-5, wherein performing the first measurement comprises performing the first measurement on a first portion of the sample, and wherein performing the second measurement comprises performing the second measurement upon a second portion of the sample that is different from the first portion of the sample.
8. The method according to inventive concept 7, wherein determining the parameter of the sample comprises determining a relationship between the first portion of the sample and second portion of the sample, based on the relationship between the bulk-level measurand and the cellular-level measurand.
9. The method according to inventive concept 7, wherein performing the second measurement upon the second portion of the sample comprises performing the second measurement upon a second portion of the sample that is diluted with respect to the first portion of the sample.
10. The method according to inventive concept 9, wherein determining the parameter of the sample comprises determining a normalization factor by determining a property of the first portion of the sample portion for using as a reference to which measurements within the second portion can be correlated.
11. The method according to inventive concept 9, wherein determining the parameter of the sample comprises determining a dilution ratio by which the second portion of the sample is diluted with respect to the first portion of the sample.
12. The method according to any one of inventive concepts 1-6, wherein the biological sample includes a blood sample, and wherein determining the parameter of the sample comprises determining a parameter of the blood sample.
13. The method according to inventive concept 12, wherein:
   measuring the bulk-level measurand of the sample comprises measuring hematocrit of the blood sample;
   measuring the cellular-level measurand of the sample comprises measuring mean corpuscular volume of the blood sample; and
   determining the parameter of the sample comprises determining the parameter of the sample, based on a relationship between the hematocrit and the mean corpuscular volume.
14. The method according to inventive concept 12, wherein:
   measuring the bulk-level measurand of the sample comprises measuring hemoglobin concentration within at least a portion of the blood sample;
   measuring the cellular-level measurand of the sample comprises measuring mean corpuscular hemoglobin of the blood sample; and
   determining the parameter of the sample comprises determining the parameter of the sample, based on a relationship between the hemoglobin concentration and the mean corpuscular hemoglobin.
15. Apparatus for use with a biological sample, the apparatus comprising:
   at least one computer processor configured to:
      measure a bulk-level measurand of the sample, by performing a first measurement on the sample,
      measure a cellular-level measurand of the sample, by performing a second measurement on the sample, and
      determine a parameter of the sample, based on a relationship between the bulk-level measurand and the cellular-level measurand.
16. A computer software product, for use with a biological sample, the computer software product comprising a non-transitory computer-readable medium in which program instructions are stored, which instructions, when read by a computer cause the computer to perform the steps of:
   measuring a bulk-level measurand of the sample, by performing a first measurement on the sample;
   measuring a cellular-level measurand of the sample, by performing a second measurement on the sample; and
   determining a parameter of the sample, based on a relationship between the bulk-level measurand and the cellular-level measurand.
17. A method for use with a biological sample, the method comprising:
   performing first and second optical measurements on the sample, using one or more optical measurement devices under respective sets of measuring conditions that are different from each other;
   measuring a measurand of the sample, based upon the first optical measurement;
   measuring a measurand of the sample, based upon the second optical measurement; and
   based on a relationship between the measurand measured based upon the first optical measurement and the measurand measured based upon the second optical measurement, determining a relationship between the measuring conditions of the one or more optical measurement devices that were used to perform the first and second optical measurements.
18. The method according to inventive concept 17, wherein the biological sample includes a blood sample, and wherein performing first and second optical measurements on the sample comprises performing first and second optical measurements on the blood sample.
19. The method according to inventive concept 17, wherein:
   performing first and second optical measurements on a sample comprises performing first and second optical measurements on respective portions of the sample that are disposed in respective portions of one or more sample chambers having respective dimensions; and
   determining the relationship between the measuring conditions of the one or more optical measurement devices that were used to perform the first and second optical measurements comprises determining a relationship between dimensions of the respective portions of the one or more sample chambers.
20. The method according to inventive concept 17, wherein performing the first and second optical measurements on the sample comprises performing at least one of the first and second optical measurements by acquiring an image of at least a portion of the sample, and wherein determining the relationship between the measuring conditions of the one or more optical measurement devices that were used to perform the first and second optical measurements comprises determining a field of view of the image.
21. The method according to inventive concept 17, wherein performing the first and second optical measurements on the sample comprises performing at least one of the first and second optical measurements by acquiring an image of at least a portion of the sample, and wherein determining the relationship between the measuring conditions of the one or more optical measurement devices that were used to perform the first and second optical measurements comprises determining a level of magnification of the image.
22. The method according to inventive concept 17, wherein:
   measuring the measurand of the sample, based upon the first optical measurement comprises measuring a given measurand of the sample, based upon the first optical measurement;
   measuring the measurand of the sample, based upon the second optical measurement comprises measuring the same given measurand of the sample, based upon the second optical measurement; and
   determining the relationship between the measuring conditions of the one or more optical measurement devices that were used to perform the first and second optical measurements comprises determining the relationship between the measuring conditions of the one or more optical measurement devices that were used to perform the first and second optical measurements, based upon a relationship the given measurand as measured based upon the first optical measurement, and the given measurand as measured based upon the second optical measurement.
23. The method according to inventive concept 17, wherein performing the first optical measurement comprises performing the first optical measurement using a given optical measurement device, and performing the second optical measurement comprises performing the second optical measurement using the same given optical measurement device.
24. The method according to any one of inventive concepts 17-22, wherein performing the first optical measurement comprises performing the first optical measurement using a first optical measurement device, and performing the second optical measurement comprises performing the second optical measurement using a second optical measurement device that is different from the first optical measurement device.
25. The method according to inventive concept 24, wherein:
   performing the first optical measurement comprises performing the first optical measurement using a first optical measurement device that is configured to measure a parameter of one or more components within the sample, the parameter being selected from the group consisting of: optical absorption, transmittance, fluorescence, and luminescence; and
   performing the second optical measurement comprises performing the second optical measurement using a microscope configured to acquire a microscopic image of the sample.
26. The method according to any one of inventive concepts 17-21 or 23, wherein:
   measuring the measurand of the sample, based upon the first optical measurement comprises measuring a first measurand of the sample, based upon the first optical measurement; and
   measuring the measurand of the sample, based upon the second optical measurement comprises measuring a second measurand of the sample that is different from the first measurand, based upon the second optical measurement; and
   determining the relationship between the measuring conditions of the one or more optical measurement devices that were used to perform the first and second optical measurements comprises determining the relationship between the measuring conditions of the one or more optical measurement devices that were used to perform the first and second optical measurements, based upon a relationship between the first and second measurands.
27. The method according to inventive concept 26, wherein measuring the first measurand comprises measuring a bulk-level measurand of the sample, and measuring the second measurand comprises measuring a cellular-level measurand of the sample.
28. Apparatus for use with a biological sample, the apparatus comprising:
   at least one computer processor configured to:
      perform first and second optical measurements on the sample, using one or more optical measurement devices under respective sets of measuring conditions that are different from each other,
      measure a measurand of the sample, based upon the first optical measurement,
      measure a measurand of the sample, based upon the second optical measurement, and
      based on a relationship between the measurand measured based upon the first optical measurement and the measurand measured based upon the second optical measurement, determine a relationship between the measuring conditions of the one or more optical measurement devices that were used to perform the first and second optical measurements.
29. A computer software product, for use with a biological sample, the computer software product comprising a non-transitory computer-readable medium in which program instructions are stored, which instructions, when read by a computer cause the computer to perform the steps of:
   performing first and second optical measurements on a sample, using one or more optical measurement devices under respective sets of measuring conditions that are different from each other;
   measuring a measurand of the sample, based upon the first optical measurement;
   measuring a measurand of the sample, based upon the second optical measurement; and
   based on a relationship between the measurand measured based upon the first optical measurement and the measurand measured based upon the second optical measurement, determining a relationship between the measuring conditions of the one or more optical measurement devices that were used to perform the first and second optical measurements.
30. A method for use with a biological sample, the method comprising:
   performing first and second optical measurements on the sample, using one or more optical measurement devices under respective sets of measuring conditions that are different from each other;
   measuring a measurand of the sample, based upon the first optical measurement;
   measuring a measurand of the sample, based upon the second optical measurement;
   normalizing the measurand measured based upon the first optical measurement and the measurand measured based upon the second optical measurement, with respect to each other; and
   determining a parameter of the sample based upon at least one of the normalized measurand measured based upon the first optical measurement and the normalized measurand measured based upon the second optical measurement.
31. Apparatus for use with a biological sample, the apparatus comprising:
   at least one computer processor configured to:
      perform first and second optical measurements on the sample, using one or more optical measurement devices under respective sets of measuring conditions that are different from each other,
      measure a measurand of the sample, based upon the first optical measurement,
      measure a measurand of the sample, based upon the second optical measurement,
      normalize the measurand measured based upon the first optical measurement and the measurand measured based upon the second optical measurement, with respect to each other, and
      determine a parameter of the sample based upon at least one of the normalized measurand measured based upon the first optical measurement and the normalized measurand measured based upon the second optical measurement.
32. A computer software product, for use with a biological sample, the computer software product comprising a non-transitory computer-readable medium in which program instructions are stored, which instructions, when read by a computer cause the computer to perform the steps of:
   performing first and second optical measurements on a sample, using one or more optical measurement devices under respective sets of measuring conditions that are different from each other;
   measuring a measurand of the sample, based upon the first optical measurement;
   measuring a measurand of the sample, based upon the second optical measurement;
   normalizing the measurand measured based upon the first optical measurement and the measurand measured based upon the second optical measurement, with respect to each other; and
   determining a parameter of the sample based upon at least one of the normalized measurand measured based upon the first optical measurement and the normalized measurand measured based upon the second optical measurement.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for use with a first portion of a bodily sample and a second portion of the bodily sample that is diluted with respect to the first portion of the bodily sample, the method comprising:
measuring a measurand within the first portion of the bodily sample;
measuring relative amounts of first and second components within the second portion of the bodily sample; and
determining a parameter of the bodily sample based upon the measurand within the first portion of the bodily sample, and the relative amounts of first and second components within the second portion of the bodily sample.

2. The method according to claim 1, wherein measuring relative amounts of first and second components within the second portion of the bodily sample comprises analyzing a microscopic image of the second portion of the bodily sample.

3. The method according to claim 1, wherein the bodily sample includes a blood sample, and measuring relative amounts of first and second components within the second portion of the bodily sample comprises measuring relative amounts of at least two components within the second portion of the blood sample, the two components being selected from the group consisting of: all white blood cell types, neutrophils, eosinophils, basophils, lymphocytes, monocytes, and white blood cell precursors.

4. The method according to claim 1, wherein the bodily sample includes a blood sample, and wherein measuring relative amounts of first and second components within the second portion of the bodily sample comprises measuring relative amounts of at least two components within the second portion of the blood sample, the two components being selected from the group consisting of: red blood cells, reticulocytes, intracellular bodies, red blood cells having a given morphology, and Howell-Jolly bodies.

5. The method according to claim 1, wherein the bodily sample includes a blood sample, and wherein measuring relative amounts of first and second components within the second portion of the bodily sample comprises measuring relative amounts of given types of platelets within the second portion of the blood sample.

6. The method according to claim 1, wherein measuring the measurand within the first portion of the bodily sample comprises measuring an absolute count of cells of a given type within the first portion of the bodily sample.

7. The method according to claim 1, wherein measuring the measurand within the first portion of the sample comprises measuring a concentration of a given component within the first portion of the bodily sample.

8. The method according to claim 1, wherein measuring the measurand within the first portion of the sample comprises performing a bulk-level measurement upon the first portion of the bodily sample.

9. The method according to claim 8, wherein performing the bulk-level measurement upon the first portion of the bodily sample comprises measuring a parameter of the first portion of the bodily sample selected from the group consisting of: optical absorption, transmittance, fluorescence, and luminescence.

10. The method according to claim 1, wherein:
measuring the measurand within the first portion of the bodily sample comprises measuring a concentration of the first component within the first portion of the bodily sample, and
determining the parameter of the bodily sample comprises determining a concentration of the second component within the first portion of the bodily sample based upon the measurand within the first portion of the bodily sample, and the relative amounts of first and second components within the second portion of the bodily sample.

11. The method according to claim 10, wherein:
the bodily sample includes a blood sample,
measuring the concentration of the first component within the first portion of the bodily sample comprises measuring a concentration of red blood cells within the first portion of the bodily sample, and
determining the concentration of the second component within the first portion of the bodily sample comprises determining a concentration of a component selected from the group consisting of: reticulocytes, intracellular bodies, red blood cells having a given morphology, and Howell-Jolly bodies, based upon the concentration of red blood cells within the first portion of the bodily sample and relative amounts of red blood cells and the selected component within the second portion of the blood sample.

12. Apparatus for use with a first portion of a bodily sample and a second portion of the bodily sample that is diluted with respect to the first portion of the bodily sample, the apparatus comprising:
at least one computer processor configured to:
measure a measurand within the first portion of the bodily sample,
measure relative amounts of first and second components within the second portion of the bodily sample, and
determine a parameter of the bodily sample based upon the measurand within the first portion of the bodily sample, and the relative amounts of first and second components within the second portion of the bodily sample.

13. The apparatus according to claim 12, wherein the at least one computer processor is configured to measure relative amounts of first and second components within the second portion of the bodily sample by analyzing a microscopic image of the second portion of the bodily sample.

14. The apparatus according to claim 12, wherein the bodily sample includes a blood sample, and wherein the at least one computer processor is configured to measure relative amounts of first and second components within the second portion of the bodily sample by measuring relative amounts of at least two components within the second portion of the blood sample, the two components being selected from the group consisting of: all white blood cell types, neutrophils, eosinophils, basophils, lymphocytes, monocytes, and white blood cell precursors.

15. The apparatus according to claim 12, wherein the bodily sample includes a blood sample, and wherein the at least one computer processor is configured to measure relative amounts of first and second components within the second portion of the bodily sample by measuring relative amounts of at least two components within the second portion of the blood sample, the two components being selected from the group consisting of: red blood cells, reticulocytes, intracellular bodies, red blood cells having a given morphology, and Howell-Jolly bodies.

16. The apparatus according to claim 12, wherein the bodily sample includes a blood sample, and wherein the at least one computer processor is configured to measure relative amounts of first and second components within the second portion of the bodily sample by measuring relative amounts of given types of platelets within the second portion of the blood sample.

17. The apparatus according to claim 12, wherein the at least one computer processor is configured to measure the measurand within the first portion of the sample by measuring an absolute count of cells of a given type within the first portion of the bodily sample.

18. The apparatus according to claim 12, wherein the at least one computer processor is configured to measure the measurand within the first portion of the sample by measuring a concentration of a given component within the first portion of the bodily sample.

19. The apparatus according to claim 12, wherein the at least one computer processor is configured to measure the measurand within the first portion of the sample by performing a bulk-level measurement upon the first portion of the bodily sample.

20. The apparatus according to claim 19, wherein the at least one computer processor is configured to perform the bulk-level measurement upon the first portion of the bodily sample by measuring a parameter of the first portion of the bodily sample selected from the group consisting of: optical absorption, transmittance, fluorescence, and luminescence.

21. The apparatus according to claim 12, wherein the at least one computer processor is configured to:
measure the measurand within the first portion of the bodily sample by measuring a concentration of the first component within the first portion of the bodily sample, and
determine the parameter of the bodily sample by determining a concentration of the second component within the first portion of the bodily sample based upon the measurand within the first portion of the bodily sample, and the relative amounts of first and second components within the second portion of the bodily sample.

22. The apparatus according to claim 21, wherein:
the bodily sample includes a blood sample,
the at least one computer processor is configured to measure the concentration of the first component within the first portion of the bodily sample by measuring a concentration of red blood cells within the first portion of the bodily sample, and
the at least one computer processor is configured to determine the concentration of the second component within the first portion of the bodily sample by determining a concentration of a component selected from the group consisting of: reticulocytes, intracellular bodies, red blood cells having a given morphology, and Howell-Jolly bodies, based upon the concentration of red blood cells within the first portion of the bodily sample and relative amounts of red blood cells and the selected component within the second portion of the blood sample.

23. A computer software product, for use with a bodily sample, the computer software product comprising a non-transitory computer-readable medium in which program instructions are stored, which instructions, when read by a computer cause the computer to perform the steps of:
measuring a measurand within a first portion of a bodily sample;
measuring relative amounts of first and second components within a second portion of the bodily sample that is diluted relative to the first portion of the bodily sample; and
determining a parameter of the bodily sample based upon the measurand within the first portion of the bodily sample and the relative amounts of first and second components within the second portion of the bodily sample.
